(19) United States
(12) Patent Application Publication
(10) Pub. No.: US 2025/0017772 A1
(43) Pub. Date: Jan. 16, 2025

(54) METHOD AND SYSTEM FOR EVALUATING PERFORMANCE OF INTRA OCULAR PRESSURE SENSORS AND MAINTENANCE SYSTEMS

(71) Applicant: JOHNSON & JOHNSON SURGICAL VISION, INC., Irvine, CA (US)

(72) Inventors: Vadim Gliner, Haifa (IL); Eran Haas, Haifa (IL); Assaf Govari, Haifa (IL); Adam Walter Toner, Ladera Ranch, CA (US)

(21) Appl. No.: 18/221,340

(22) Filed: Jul. 12, 2023

(51) Int. Cl. A61F 9/007

(52) U.S. Cl. CPC A61F 9/00745; A61B 2017/00128; A61B 2090/064

(57) ABSTRACT

A system, method and computer program product, the system comprising: a phacoemulsification probe having a needle at its distal end, the needle configured to be inserted into an eye of a patient; a pressure sensor, and a processor, configured to repeatedly: obtain a plurality of pressure values taken by the pressure sensor, each of the plurality of pressure values indicating an intraocular pressure (IOP) measurement taken during a surgical procedure; calculate an overall measurement for the surgical procedure based on the pressure values; and displaying on a display device a graphic representation of the overall measurement.

### FIELD OF THE DISCLOSURE

This disclosure relates generally to a method for relaying information obtained from an intra ocular pressure (IOP) sensor to a user, and specifically to a method for evaluating the performance of IOP maintenance or sensing systems.

### BACKGROUND OF THE DISCLOSURE

Many types of intra ocular operations require the stabilization of the intraocular pressure (IOP) within a predetermined range, such as 20-200 mmHg.

One particular operation type is a cataract removal operation. A cataract is a clouding and hardening of the eye's natural lens, which often happens when people get older. A common treatment of cataract is phacoemulsification cataract surgery. In the procedure, a portion of the anterior surface of the lens capsule is removed to gain access to the cataract. The surgeon then uses a phacoemulsification probe, which is an ultrasonic handpiece with a needle. The tip of the needle vibrates at ultrasonic frequency, which emulsifies the cataract lens. At a same time, a pump aspirates particles and fluid from the eye through the tip, wherein the aspirated fluids are replaced with irrigation of a balanced salt solution to maintain the intraocular pressure in the anterior chamber of the eye. After removing the cataract with phacoemulsification, the softer outer lens cortex is removed with suction. An intraocular lens is then introduced into the empty lens capsule restoring the patient's vision.

Due to the combination of irrigation and aspiration involved in the procedure, maintaining the IOP in cataract operations is particularly important but also challenging. Therefore, there is a need for a method and system for monitoring the IOP, in particular but not only in phacoemulsification operations.

### BRIEF DESCRIPTION OF THE DRAWINGS

The present disclosure will be more fully understood from the following detailed description of the examples thereof, taken together with the drawings, in which:

FIG. 1A is a schematic, pictorial view of a phacoemulsification apparatus, in accordance with an example of the present disclosure;

FIG. 1B is a schematic view of an ophthalmic curette device comprising a pressure sensor, which can be used in accordance with some exemplary examples of the disclosure;

FIG. 1C is a schematic view of a pressure sensor, which can be used in accordance with some exemplary examples of the disclosure;

FIG. 2 is an illustration of an exemplary IOP histogram, in accordance with some exemplary examples of the disclosure;

FIG. 3 is an illustration of another exemplary IOP histogram, in accordance with some exemplary examples of the disclosure;

FIG. 4 is a flowchart of steps in a method for displaying pressure information to a user of a phacoemulsification system, in accordance with some exemplary examples of the disclosure; and

FIG. 5 is a block diagram of a computing platform for determining and displaying representations of IOP, in accordance with some exemplary examples of the disclosure.

### DETAILED DESCRIPTION OF EXAMPLES

In the following description, numerous specific details are set forth in order to provide a thorough understanding of the present invention. It will be apparent to one skilled in the art, however, that the present invention may be practiced without these specific details. In other instances, well-known circuits, control logic, and the details of computer program instructions for conventional algorithms and processes have not been shown in detail in order not to obscure the present invention unnecessarily.

Software programming code, which embodies aspects of the present invention, is typically maintained in permanent storage, such as a computer readable medium. In a client-server environment, such software programming code may be stored on a client or a server. The software programming code may be embodied on any of a variety of known media for use with a data processing system. This includes, but is not limited to, magnetic and optical storage devices such as disk drives, magnetic tape, compact discs (CD's), digital video discs (DVD's), and computer instruction signals embodied in a transmission medium with or without a carrier wave upon which the signals are modulated. For example, the transmission medium may include a communications network, such as the Internet. In addition, while the invention may be embodied in computer software, the functions necessary to implement the invention may alternatively be embodied in part or in whole using hardware components such as application-specific integrated circuits or other hardware, or some combination of hardware components and software.

In the description below, the term “about” as related to numerical values may include values that are in the range of +/−10% of the indicated value.

### Overview

A cataract is often handled by a phacoemulsification procedure, in which a needle of an ultrasonic handpiece is inserted into a patient's eye, wherein the needle can vibrate at ultrasonic frequency, to emulsify the cataract.

During the operation, a pump aspirates particles and fluid from the eye through the tip. An irrigation system irrigates a balanced salt solution to compensate for the aspirated material and maintain the IOP. The aspiration and irrigation may be coordinated such that the IOP in the anterior chamber of the eye is maintained as stable as possible and within a predetermined range.

A pressure sensor may be employed to measure or assess the pressure such that it can be maintained accurately.

The pressure sensor may be combined with the aspiration and/or irrigation systems, or may be implemented as an independent system. In any case, the irrigation and/or aspiration system may be controlled in accordance with the sensed and required pressure.

However, it may also be important for a user such as a physician, a supervisor, a developer of a new phacoemulsification system or a new pressure sensor, or the like to inspect the pressure during the operation. For example, too high or too low instantaneous pressure may be acceptable although undesirable, but not over longer periods of time.

For example, when testing a new or an upgrade of an irrigation system, aspiration system or pressure sensor, it may be important to monitor the pressure and verify that the tested system is operating correctly, and the required pressure is maintained for at least a predetermined portion of the time.

In further examples, when training or evaluating a new user or an experienced user with a new system, it may also be required to monitor the IOP, in order to verify that the user is operating the system correctly.

In addition to the ongoing momentary pressure, it may also be required to obtain a general view of the pressure throughout the operation, and determine whether the IOP is within the required or desired range for a sufficient or acceptable portion of the operation duration.

Thus, in accordance with some examples of the disclosure, a holistic assessment of the pressure throughout the operation may be generated upon instantaneous readings received from a pressure sensor, and displayed over a display device associated with the system.

In some exemplary examples, a histogram of the pressure may be generated and displayed, such that a single glance may be sufficient to verify whether the pressure was within a desired range a significant part of the time. The histogram may be divided into a number of bins/bars having equal widths, wherein at least one bar includes a desired pressure level, and other bars may include higher or lower pressure levels. If the central bars, representing pressure levels in the vicinity of the desired levels are significantly higher than the peripheral bars, it may be determined that the IOP is maintained in the required range. If the peripheral bars are higher than the central ones, this may indicate incorrect and/or unstable pressure, which is less desirable. The histogram may be displayed during the operation and updated periodically, and also once the operation is completed.

In some examples, in addition to the accumulated histogram, the display may also indicate a current pressure on the histogram. In some examples, during or after the operation, a histogram related to only a portion of the time of the operation may be displayed. For example, a histogram of the first half or the second half of the operation may be displayed, or the two histograms may be displayed side by side for comparison.

In some examples, whether the factor to be evaluated is the irrigation or aspiration system or controller, the pressure sensor, or the user, a total score for the obtained pressure may be calculated and displayed. For example, the mean value and the standard deviation of the histogram may be presented, wherein the mean value should be as close as possible to the desired value or within an allowable distance therefrom, and the standard deviation should be as small as possible.

In some examples, two or more histograms representing measures taken by two or more sensors may be generated and displayed, for example side by side, such that one sensor may serve as a gold standard, or ground truth, and the other may be evaluated against it.

In further examples, two or more histograms representing measures taken during two or more operations may be generated and displayed, for comparison of a tested factor.

### System Description

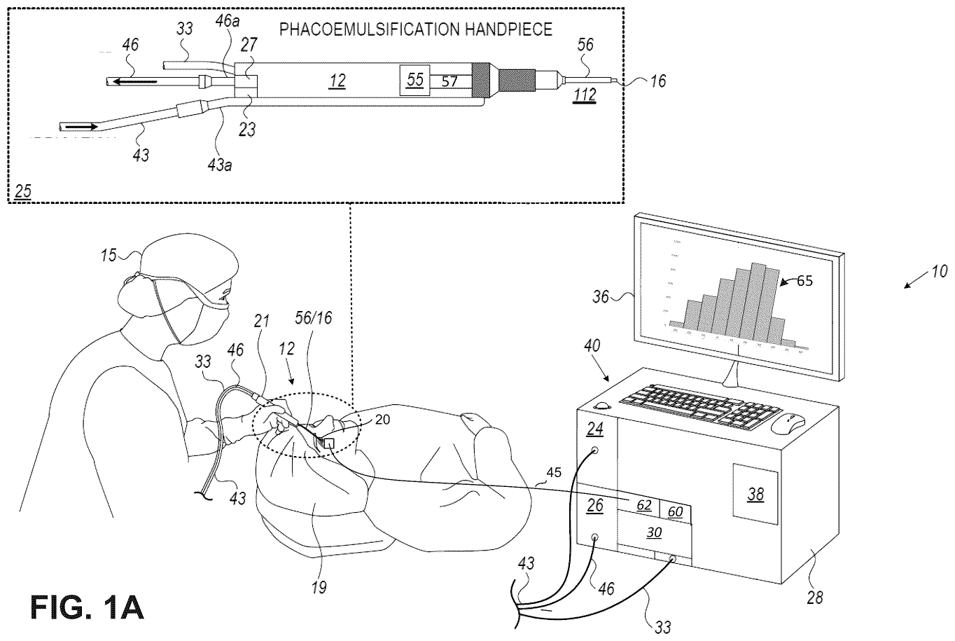

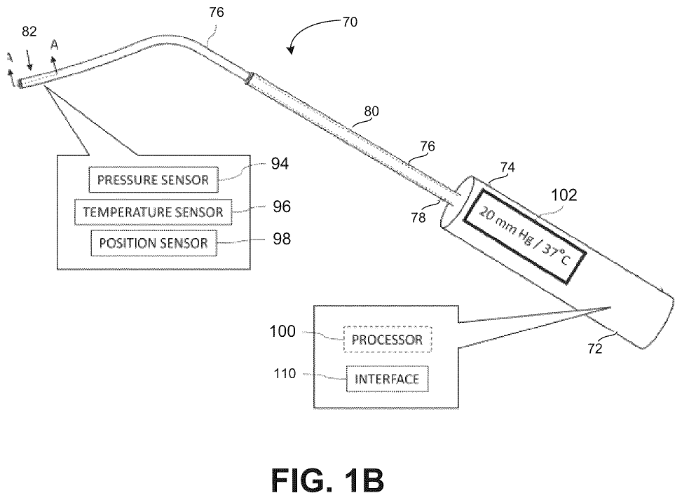

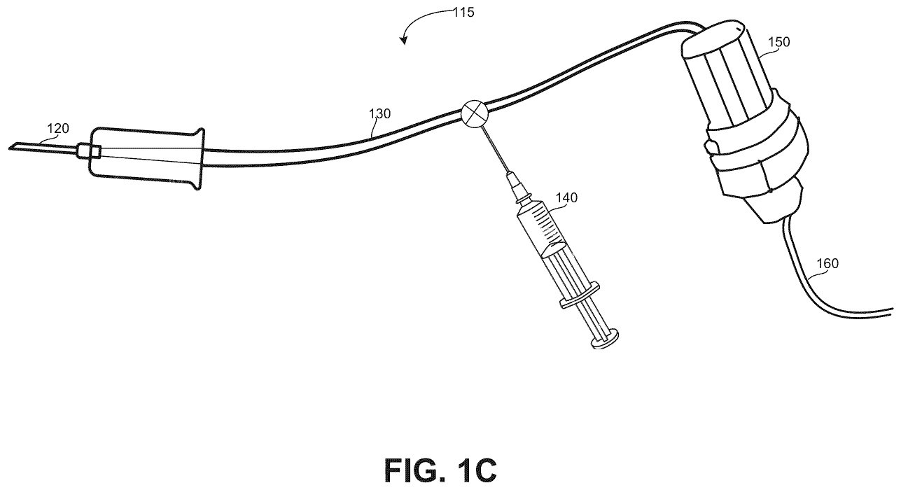

Referring now to FIG. 1, showing an exemplary phacoemulsification apparatus 10, which includes a phacoemulsification probe 12 having at its distal end 112 a needle 16 which may comprise an optical fiber therein. Needle 16 is configured to be inserted by a physician 15 into the lens capsule of an eye of a patient 19 to remove a cataract lens. While needle 16 is shown in inset 25 as a straight object, it is appreciated that any suitable needle may be used with phacoemulsification probe 12, for example, a curved or bent tip needle commercially available from Johnson & Johnson Surgical Vision, Inc., Irvine, CA, USA.

System 10 may comprise console 28, comprising a user interface 40, including physical and virtual controls such as a keyboard, a mouse, a touchscreen, a joystick, a foot pedal, a speaker, a microphone, or others, for inputting data or commands to the apparatus or receiving data from the apparatus, and a processor 38.

In an example, system 10 may comprise a display 36 for displaying various images and aspects of the operation to the physician. In some examples, one or more controls of user interface 40 and display 36 may be integrated into a touch screen graphical user interface.

Probe 12 may comprise ultrasound transducer 55, e.g., a piezoelectric ultrasound transducer, which is configured to vibrate horn 57 and needle 16 in one or more resonant vibration modes of the combined horn and needle element. During the phacoemulsification procedure, upon the application of one or more drive signals to ultrasound transducer 55, the vibration of needle 16 is used to emulsify the cataract. Ultrasound transducer 55 and horn 57, or different combinations providing the same effect are collectively referred to as an ultrasound transducer.

In some examples, eye fluid and waste matter (e.g., emulsified parts of the cataract) are aspirated via a lumen in needle 16 to a collection receptacle (not shown) by an aspiration pump 26, which may be controlled by processor 38, using aspiration tubing line 46 running from aspiration channel 46a of probe 12 to console 28.

In some examples, probe 12 may further comprise a coaxial irrigation sleeve 56 that at least partially surrounds needle 16. During the phacoemulsification procedure, an irrigation pump 24 which may be controlled by processor 38 may pump irrigation fluid from an irrigation reservoir (not shown) to irrigation sleeve 56, to irrigate the eye. The fluid may be pumped via an irrigation tubing line 43 running from console 28 to an irrigation channel 43a of probe 12.

In some examples, irrigation pump 24 and aspiration pump 26 may be controlled by processor 38 in accordance with readings received from irrigation sensor 23 and aspiration sensor 27, respectively, to maintain the IOP within predetermined limits. An overall pressure within the eye chamber may be assessed, for example by processor 38, based on the readings received from irrigation sensor 23 and aspiration sensor 27.

In further examples, the system may comprise a pressure sensor for sensing the overall pressure within the eye chamber. The pressure sensor may be comprised within probe 12, or be an external unit connectable to console 28 or to a processing unit.

Processor 38 is thus adapted to control the operation of various functions of probe 12 such as the irrigation and aspiration, in accordance with the physician's commands as provided via user interface 40, and with various measurements.

In some examples, processor 38 may automatically control the irrigation and aspiration in order to maintain a required pressure level within the chamber.

It is appreciated that some or all of the functions of processor 38 may be combined in a single physical component. Alternatively, processor 38 may be implemented using multiple physical components. These physical components may comprise hard-wired or programmable devices, or a combination thereof. In some examples, at least some of the functions of processor 38 may be carried out by suitable software stored in a memory device of processor 38 or console 28. This software may be downloaded to a device in electronic form, over a network, or the like. Alternatively, or additionally, the software may be stored in tangible, non-transitory computer-readable storage media, such as optical, magnetic, or electronic memory.

The system may comprise drive module 30 for activating ultrasound transducer 55, for example setting the operation parameters and supplying current to ultrasound transducer 55. Drive module 30 may be realized in hardware or software, for example, in a proportional-integral-derivative (PID) control architecture.

In some examples, display 36 may also display information related to the momentary measurements of the IOP or a more general view thereof, such as a histogram 65, textual information, or the like.

FIG. 1B and FIG. 1C below show pictorial views of devices comprising pressure sensors, which may be used as devices external to a phacoemulsification system, in accordance with some examples of the disclosure.

Referring now to FIG. 1B, showing a partly pictorial, partly block diagram view of an ophthalmic curette device 70 comprising a pressure sensor, constructed and operative in accordance with an example of the present disclosure. Ophthalmic curette device 70 includes a handle 72 having a distal end 74. Handle 72 may be formed from any suitable material or combination of materials, for example, a metal and/or polymer.

Ophthalmic curette device 70 includes a rigid biocompatible tube 76 having a proximal end 78 coupled with distal end 74 of handle 72. Tube 76 may have any suitable form. In some examples, tube 76 is straight. In other examples, tube 76 is bent or curved to any suitable angle. Tube 76 may be formed from any suitable metal, for example, stainless steel or titanium. In some examples, tube 76 may be replaced by a tube of any suitable material, for example, a polymer such as PEEK or FEP. Suitable metal and polymer tubing are commercially available from IDEX corporation, Lake Forest, Ill., USA.

A proximal portion of tube 76 is optionally surrounded by an elongated supporting member 80 to provide additional support to the proximal portion which is not inserted into an eye. Supporting member 80 may be formed from any suitable material, for example, a metal such as stainless steel or titanium, or a polymer. Tube 76 has a distal end 82.

Device 70 may be inserted into a chamber of the eye of patient 19 in addition to phacoemulsification probe 12. In some examples, device 10 and probe 12 may be inserted into the eye through two incisions.

Tube 76 (which extends beyond the supporting member 80) may have any suitable length, which is long enough for tube 76 to be inserted into the eye and be maneuvered during the medical procedure. In some examples, tube 76 may have a minimum length of 2 cm. Tube 76 may have any suitable outer diameter and wall thickness. In some examples, tube 76 has an outer diameter between 0.1 mm and 0.8 mm, and a wall thickness between 0.03 mm and 0.2 mm.

In some examples, device 70 may include a pressure sensor 94 disposed inside tube 76 at distal end 82 of tube 76. Pressure sensor 94 is configured to provide a signal responsive to the IOP inside the eye chamber. Any suitable pressure sensor, which is small enough to be inserted into tube 76 and is sensitive enough to accurately measure the intraocular pressure, may be used. A suitable pressure sensor (e.g., Novasensor P330 series absolute pressure sensor die) is commercially available from Amphenol Thermometrics Inc., St. Mary's, Pa. 15857, United States. The Novasensor P330 series absolute pressure sensor die has high pressure sensitivity (standard pressure range 450 to 1050 mmHg) and has a 330×180 microns cross section. The longest dimension of the P330 pressure sensor may be aligned parallel to the axis of tube 76.

In some examples, device 70 may include additional sensors, such as but not limited to temperature sensor 96 and position sensor 98, such as a magnetic position sensor. Magnetic position sensor 98 may be configured to be used as part of a position tracking system (not shown) for tracking a position of distal end 82 of tube 76. As tube 76 is rigid, magnetic position sensor 98 may be disposed at any suitable position inside tube 76.

Device 70 may include a processor 100 and a display 102. In some examples, processor 100 is configured to compute a pressure value responsive to the signal provided by pressure sensor 94. Display 102 may be configured to render the pressure value and/or other measures such as a temperature value. Processor 100 and display 102 may be disposed in/on handle 72. Display 102 may include any suitable display, for example, a liquid crystal display.

Additionally or alternatively, the data including the pressure measure may be transmitted to console 28 and displayed on a display device such as display 36 of FIG. 1A. Alternatively, the data may be displayed on any remote console coupled with handle 72 wirelessly and/or via wires 108, and configured to render the pressure value and/or the temperature value. Handle 72 and the console may each include an interface 110 to provide data communication between handle 72 and the console. Interfaces 110 may comprise circuitry to allow wired and/or wireless communication therebetween.

In some examples, some or all of the functions of processor 100 may be combined in a single physical component, or alternatively implemented using multiple physical components. These physical components may comprise hard-wired or programmable devices, or a combination of the two. In some examples, at least some of the functions of processor 100 may be carried out by a programmable processor under the control of suitable software. This software may be downloaded to a device in electronic form, over a network, for example. Alternatively, or additionally, the software may be stored in tangible, non-transitory computer-readable storage media, such as optical, magnetic, or electronic memory. In other examples, display 102 and/or processor 100 are not included in handle 72 or the console.

In some examples, the sensor signal(s) and/or pressure and/or computed temperature values are conveyed via wires and/or wirelessly to a remote processing device (not shown) which processes the sensor signals and/or uses the computed pressure and/or temperature values as part of a medical procedure process. For example, medical procedure parameters such as phacoemulsification probe needle vibration frequency, amplitude, and/or mode may be adjusted according to the temperature and/or pressure values.

Pressure sensor 94 may be placed so that when the tube is inserted into the eye, the pressure sensor is also inside the eye and exposed to the eye fluid. In some examples, the pressure sensor is coated with a waterproof coating such as Parylene, silicon, or polyurethane.

Device 70 or another phacoemulsification device may be used to manipulate cataract material during a procedure. However, during a phacoemulsification procedure, readings from the pressure sensor may become compromised because emulsified material adheres to the sensing surface of the pressure sensor. Therefore, some examples of the disclosure incorporate an irrigation channel (separate from an irrigation line used for the phacoemulsification probe) into device 70. The irrigation channel is shaped, and positioned with respect to the sensing surface of the pressure sensor, to be able to direct irrigation fluid over the sensing surface to remove emulsified material. The irrigation fluid may be conveyed at any suitable rate, e.g., 2 milliliters per second or in the range of 1 to 5 milliliters per minute. In some examples, the irrigation fluid may be conveyed intermittently, or the irrigation rate may be modulated. In these examples, a signal supplied by the pressure sensor may be sampled when the irrigation fluid is not being conveyed or when the irrigation rate is below a threshold value. Sampling the signal when the irrigation fluid is not being conveyed or when the irrigation rate is below the threshold value may result in more accurate pressure measurements.

In some examples, the pressure sensor is at least partially placed in the irrigation channel, which extends through the tube to a distal tip of the tube. In some examples, the pressure sensor is at least partially placed in the distal tip.

In some examples, the tube may include a beveled opening, which extends longitudinally along all of the distal tip of the tube. The pressure sensor is at least partially placed in the distal tip. In this manner, the sensing surface of the pressure sensor is exposed directly to the fluid in the chamber of the eye while being cleaned by the flowing irrigation fluid from the irrigation channel. The beveled opening may have any suitable angle with the end of the tube. In some examples, the beveled opening defines a plane which has an angle in the range of 30 to 70 degrees with a plane perpendicular to a direction of elongation of the tube.

Referring now to FIG. 1C, showing another example of an external pressure sensor, to be used with some examples of the disclosed subject matter.

The pressure sensing device 115 may comprise a hollow needle 120 coupled with tube 130, which may be coupled on its other end with pressure sensor 150. Needle 120 may be filled with fluid prior to being inserted into the eye of the subject, such that due to pressure balancing, the pressure inside the needle is the same as the IOP. Thus, the IOP may be measured by pressure sensor 150.

However, tube 130 or needle 120 may contain air bubbles, in which case the pressure measured by pressure sensor 150 may be unindicative of the IOP, thereby the air bubbles may need to be removed. In order to avoid this situation, tube 130 and needle 120 may be irrigated, for example with fluid injected from syringe 140, to clear out particles, air bubbles, or other objects. The fluid may be injected at predetermined time intervals, whenever the pressure remains constant for over a predetermined period of time, when the pressure exceeds a predetermined range, or the like.

An example of an optional pressure sensor is provided in https://www.festo.com/us/en/a/download-document/datasheet/8000100 and is available from Festo Pneumatic Israel Ltd., of Modi'in, Israel.

The pressure values measured by pressure sensor 150 may be provided wirelessly or via wire 160 to processor 38, processor 100 or another processor. The pressure, or any processing of one or more pressure readings, may be displayed over any display device, such as display device 36.

It is appreciated that device 70 or pressure sensor device 115 may be inserted into the eye of the patient in addition to any phacoemulsification tool. The measured pressure and other views thereof, such as a histogram, may be rendered and displayed to a user as part of experimenting with new equipment, training with a new system, training of a professional, or the like.

It is also appreciated that direct measurement is advantageous over assessment of the pressure based on partial data items or complex calculations, as it is faster and thus incurs little or no delay. However, there is a tradeoff between the measurement rate and the stability of the measurements, as more frequent measurements are more subject to instantaneous fluctuations and are therefore less stable.

The operations of the phacoemulsification system, including the aspiration and irrigation, are aimed at balancing the IOP and eliminating these fluctuations as much as possible. The level of success in maintaining the balance may be reflected by the IOP histogram.

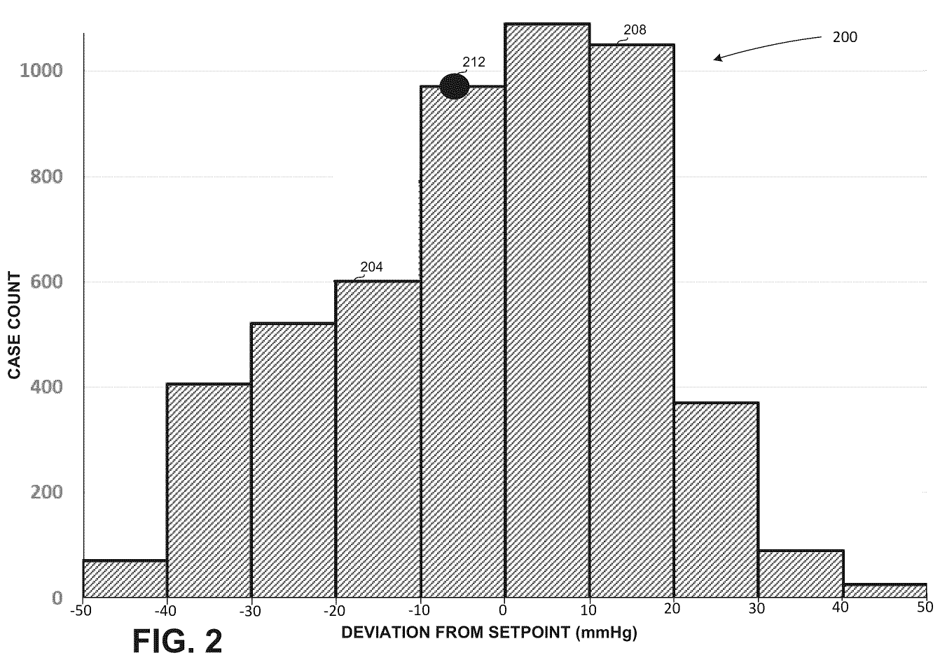

Referring now to FIG. 2, illustrating an exemplary IOP histogram, in accordance with some examples of the disclosure.

FIG. 2 shows histogram 200. The x axis represents the deviation of measurements from a pressure setpoint, measured for example in mmHg. The range of values is divided into bars, wherein in some examples the bars may be of equal width, for example 10 mmHg. The height of the column associated with each bar (y-axis) represents the number of pressure measurements (case counts) whose values are within the range associated with the bar.

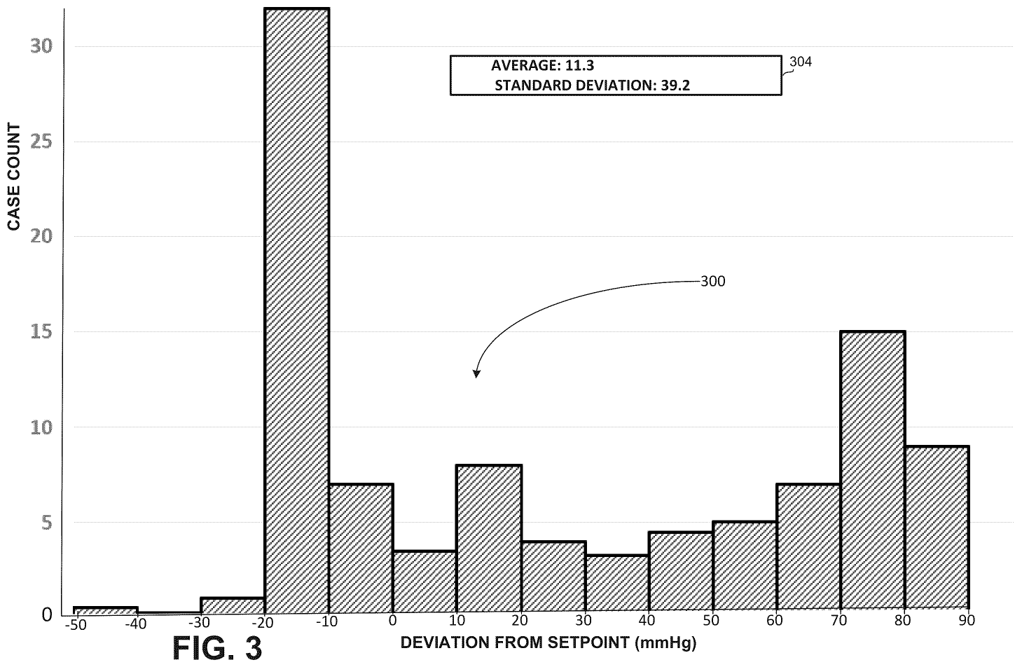

Referring now also to FIG. 3, showing another histogram 300 having the same axes, although with a smaller case count.

It is seen that histogram 200 has high concentration of cases in bars within the range of [(−10) . . . 20] mmHg deviation, while histogram 300 has high concentration of cases within the ranges of [(−20) . . . (−10)] mmHg and [70 . . . 80] mmHg.

Thus, it is clear that in the operation associated with histogram 200, the IOP was maintained, or at least the pressure sensor so reported, for a significantly larger portion of the time within a small deviation from the setpoint, than in histogram 300, in which the pressure was at significant deviation, in both directions, from the setpoint.

In some examples, an indication of a current pressure may be displayed, for example as indicator 212 of FIG. 2.

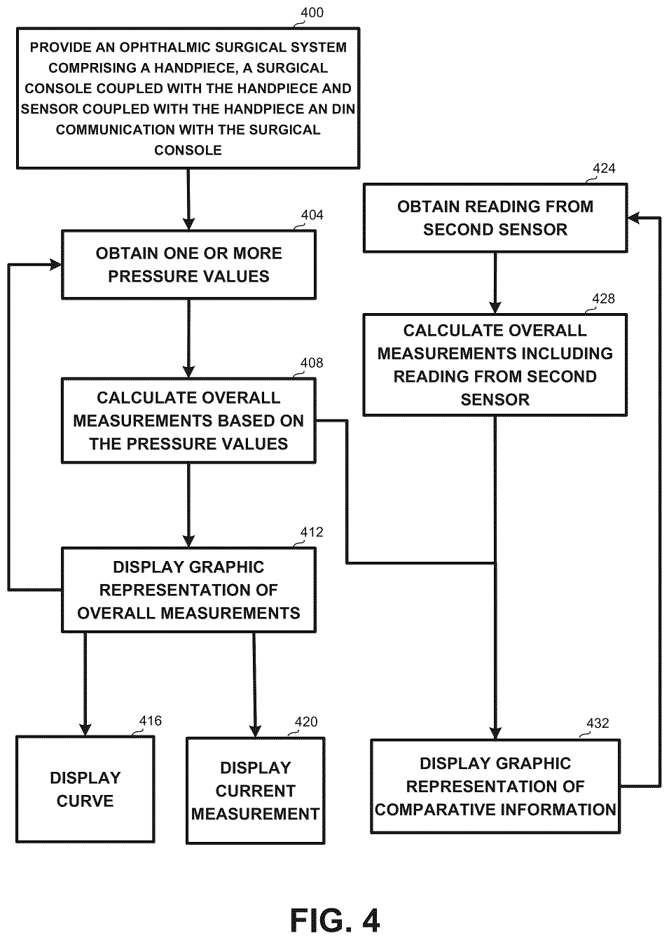

Referring now to FIG. 4, showing a flowchart of a method for displaying pressure information to a user of a phacoemulsification system, in accordance with some exemplary examples of the disclosure.

At step 400 an ophthalmic surgical system may be provided. The ophthalmic surgical system may comprise a probe, also referred to as a handpiece, a surgical console coupled with the handpiece, and one or more sensors coupled with the handpiece and in communication with the surgical console.

At step 404, one or more pressure values indicative of an IOP may be received. Each of the values may indicate an IOP value taken at a certain time. Each such measurement may be received as raw data from a pressure measurement device, or as a result of processing one or measurements by a processor.

At step 408, overall measurement may be calculated for the session based on the received values. For example, the calculation may include dividing the value range into bars and creating a histogram data structure.

At step 412, a graphic representation of the measurements may be displayed, for example, a histogram as shown in FIG. 2 or FIG. 3 above.

It is appreciated that as further readings are received at step 404, the overall measurements may be processed and updated at step 408, and the display may be updated at step 412.

In addition to displaying the overall measurements, such as the histogram, additional information may be displayed.

For example, at step 416, a curve representing the rim of the histogram may be displayed. In another example, at step 420 a current measurement may be displayed as shown for example by marker 212 of FIG. 2.

Further data or representations may also be displayed, for example as text indicating an average pressure and standard deviation as shown by text 304 of FIG. 3, or the like.

In further examples, the display may be used for comparing the readings from two sensors. Such usage may be particularly useful when evaluating a system that provides unreliable pressure measurements. Additionally, the system may be useful when used with a system that does not have pressure measurement capabilities.

In such examples, in addition to receiving and processing the pressure values from the sensor, at step 424 readings may be received from a second sensor.

At step 428, a second overall measurement may be calculated, and at step 432 a graphic representation of the two measurements may be displayed, or a comparison thereof may be displayed.

In further examples, two or more histograms of operations performed using different systems may be displayed side-by-side for comparison purposes, assuming that each histogram is representative of the performance enabled by the respective system.

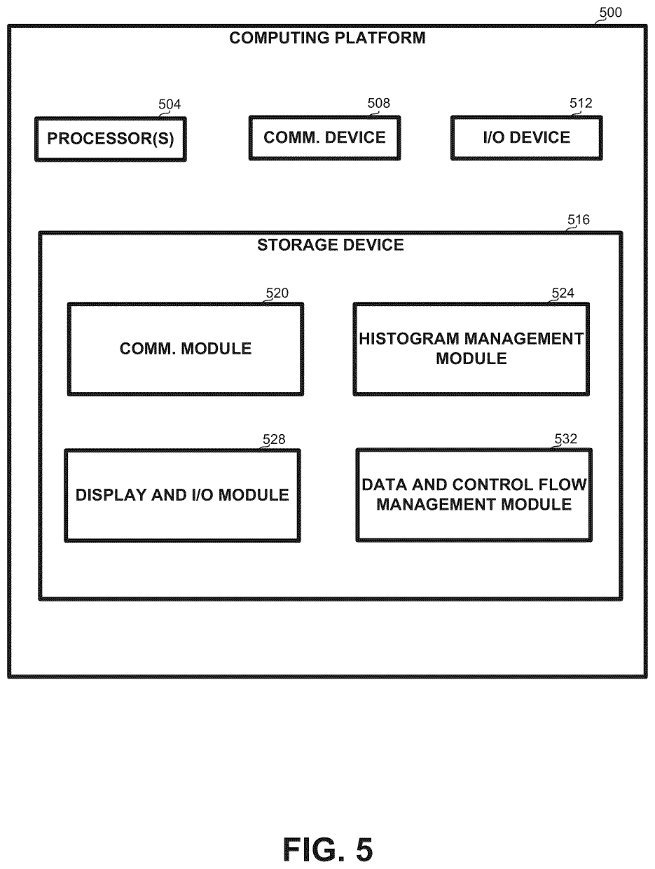

Referring now to FIG. 5, showing a block diagram of a computing platform 500 for determining and displaying representations of IOP, in accordance with some exemplary examples of the disclosure.

It is appreciated that computing platform 500 MAY be embedded within console 28, but may also be a standalone computing platform or embedded elsewhere and be in operative communication with console 28.

Computing platform 500 may be implemented as one or more computing platforms which may be operatively connected to each other. For example, one or more remote computing platforms, which may be implemented for example on a cloud computer. Other computing platforms may be a part of a computer network of the associated organization. In other examples, all the functionalities may be provided by one or more computing platforms all being a part of the organization network.

Computing platform 500 may comprise one or more processors 504 located on the same computing platform or not, which may be one or more Central Processing Units (CPU), microprocessors, electronic circuits, Integrated Circuits (IC) or the like. Processor 504 may be configured to provide the required functionality, for example by loading to memory and activating the software modules stored on storage device 516 detailed below.

Computing platform 500 may comprise a communication device 508 for communicating with other devices or other computing platforms as necessary, for example obtaining readings from one or more pressure sensors, storing data on remote storage devices, or the like. Communication module 508 may be adapted to interface with any communication channel such as Local Area Network (LAN), Wide Area Network (WAN), cellular network or the like, and use any relevant communication protocol.

Computing platform 500 may comprise an Input/Output (I/O) device 512, such as a display device, for displaying information such as histograms, comparative information, or the like. I/O device 512 may also be operative in receiving instructions from a user, for example changing the display settings. In some examples, I/O device 512 may be display 36 of FIG. 1.

Computing platform 500 may comprise a storage device 516, such as a hard disk drive, a Flash disk, a Random Access Memory (RAM), a memory chip, or the like. In some exemplary examples, storage device 516 may retain program code operative to cause processor 504 to perform acts associated with any of the modules listed below, or steps of the method of FIG. 4 above. The program code may comprise one or more executable units, such as functions, libraries, standalone programs or the like, adapted to execute instructions as detailed below.

Alternatively, or additionally, the provided instructions may be stored on non-transitory tangible computer-readable media, such as magnetic, optical, or electronic memory.

Storage device 516 may comprise communication module 520 for transmitting and receiving data to and from other components of the system or other systems through communication device 508, such as pressure readings, readings or data from other sensors, user settings, histograms, or the like.

Storage device 516 may comprise histogram management module 524 for determining bins for the histogram based on the received readings, distributing the readings into the bars and maintaining the count, designing a display of the histogram, or the like.

Storage device 516 may comprise display and I/O module 528, for rendering a display to the user to be displayed over I/O device 512, such as histograms, current readings, or the like. Display and I/O module 528 may also be operative in receiving instructions and settings from the user.

Storage device 516 may comprise data and control flow management module 532, for activating the modules above in the correct order and timing, and with the required input, for example determining or updating the histogram bars when sufficient number of readings is received, determining the histogram based on the received readings, updating the display once an updated histogram is available, or the like.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembly instructions, instruction-set-architecture instructions, machine instructions, machine dependent instructions, microcode, firmware instructions, state-setting data, or either source code or object code written in any combination of one or more programming languages, programming languages such as Java, C, C++, Python, or others. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some examples, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to examples of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general-purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various examples of the present invention. In this regard, each block in the flowchart or block diagrams may represent a module, segment, or portion of instructions, which comprises one or more executable instructions for implementing the specified logical function(s). In some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

### EXAMPLES

### Example 1

A method for evaluating eye chamber pressure stability during an ophthalmic procedure, comprising: providing an ophthalmic surgical system comprising a handpiece, a surgical console coupled with the handpiece, and at least one sensor coupled with the handpiece and in communication with the surgical console: obtaining a plurality of pressure values from the at least one sensor, each of the plurality of pressure values indicating an intraocular pressure (IOP) measurement taken during a surgical procedure: calculating an overall measurement based on the pressure values; and displaying on a display device a graphic representation of the overall measurement, wherein the overall measurement illustrates the eye chamber pressure stability.

### Example 2

The method according to example 1, wherein the overall measurement is a histogram of the pressure values taken during the operation.

### Example 3

The method according to example 1, further comprising displaying a curve representing a rim of the histogram.

### Example 4

The method according to example 1, wherein the surgical procedure is a phacoemulsification procedure.

### Example 5

The method according to example 1, further comprising displaying additional information.

### Example 6

The method according to example 5, wherein the additional information is an instantaneous pressure value.

### Example 7

The method according to example 5, wherein the additional information comprises an average pressure value.

### Example 8

The method according to example 5, wherein the additional information comprises a standard deviation of the pressure values.

### Example 9

The method according to example 1, wherein the at least one sensor is a first sensor and further comprising a second sensor located externally to the ophthalmic surgical system.

### Example 10

The method according to example 1, further comprising displaying a second overall measurement taken while using different equipment during the surgical procedure.

### Example 11

The method according to example 1, further comprising displaying a second overall measurement taken during another surgical procedure.

### Example 12

An ophthalmic surgical system, comprising: a phacoemulsification probe having a needle at its distal end, the needle configured to be inserted into an eye of a patient: a pressure sensor; and a processor, configured to repeatedly: obtain a plurality of pressure values taken by the pressure sensor, each of the plurality of pressure values indicating an intraocular pressure (IOP) measurement taken during a surgical procedure: calculate an overall measurement for the surgical procedure based on the pressure values; and displaying on a display device a graphic representation of the overall measurement.

### Example 13

The system according to example 12, wherein the pressure sensor is external to the phacoemulsification probe.

### Example 14

The system according to example 12, wherein the pressure sensor is comprised in the phacoemulsification probe.

### Example 15

The system according to example 12, wherein the overall measurement is a histogram of the pressure values taken during the operation.

### Example 16

The system according to example 12, wherein the processor is further configured to display a second overall measurement taken during the surgical procedure.

### Example 17

The system according to example 12, wherein the processor is further configured to display a second overall measurement taken during another surgical procedure.

### Example 18

A computer program product comprising a non-transitory computer readable medium retaining program instructions, which instructions when read by a processor, cause the processor to perform: obtaining a plurality of pressure values from at least one sensor coupled with a handpiece and in communication with a surgical console, the sensor, the handpiece and the surgical console associated with an ophthalmic surgical system, wherein each of the plurality of pressure values indicates an intraocular pressure (IOP) measurement taken during a surgical procedure: calculating an overall measurement based on the pressure values; and displaying on a display device a graphic representation of the overall measurement, wherein the overall measurement illustrates the eye chamber pressure stability.

It will be appreciated that the examples described above are cited by way of example, and that the present disclosure is not limited to what has been particularly shown and described hereinabove. Rather, the scope of the present disclosure includes both combinations and sub-combinations of the various features described hereinabove, as well as variations and modifications thereof which would occur to persons skilled in the art upon reading the foregoing description and which are not disclosed in the prior art.

1. A method for evaluating eye chamber pressure stability during an ophthalmic procedure, comprising:
providing an ophthalmic surgical system comprising a handpiece, a surgical console coupled with the handpiece, and at least one sensor coupled with the handpiece and in communication with the surgical console;
obtaining a plurality of pressure values from the at least one sensor, each of the plurality of pressure values indicating an intraocular pressure (IOP) measurement taken during a surgical procedure;
calculating an overall measurement based on the pressure values; and
displaying on a display device a graphic representation of the overall measurement, wherein the overall measurement illustrates the eye chamber pressure stability.

2. The method of claim 1, wherein the overall measurement is a histogram of the pressure values taken during the operation.

3. The method of claim 2, further comprising displaying a curve representing a rim of the histogram.

4. The method of claim 1, wherein the surgical procedure is a phacoemulsification procedure.

5. The method of claim 1, further comprising displaying additional information.

6. The method of claim 5, wherein the additional information is an instantaneous pressure value.

7. The method of claim 5, wherein the additional information comprises an average pressure value.

8. The method of claim 5, wherein the additional information comprises a standard deviation of the pressure values.

9. The method of claim 1, wherein the at least one sensor is a first sensor and further comprising a second sensor located externally to the ophthalmic surgical system.

10. The method of claim 1, further comprising displaying a second overall measurement taken while using different equipment during the surgical procedure.

11. The method of claim 1, further comprising displaying a second overall measurement taken during another surgical procedure.

12. An ophthalmic surgical system, comprising:
a phacoemulsification probe having a needle at its distal end, the needle configured to be inserted into an eye of a patient;
a pressure sensor; and
a processor, configured to repeatedly:
obtain a plurality of pressure values taken by the pressure sensor, each of the plurality of pressure values indicating an intraocular pressure (IOP) measurement taken during a surgical procedure;
calculate an overall measurement for the surgical procedure based on the pressure values; and
displaying on a display device a graphic representation of the overall measurement.

13. The system of claim 12, wherein the pressure sensor is external to the phacoemulsification probe.

14. The system of claim 12, wherein the pressure sensor is comprised in the phacoemulsification probe.

15. The system of claim 12, wherein the overall measurement is a histogram of the pressure values taken during the operation.

16. The system of claim 12, wherein the processor is further configured to display a second overall measurement taken during the surgical procedure.

17. The system of claim 12, wherein the processor is further configured to display a second overall measurement taken during another surgical procedure.

18. A computer program product comprising a non-transitory computer readable medium retaining program instructions, which instructions when read by a processor, cause the processor to perform:
obtaining a plurality of pressure values from at least one sensor coupled with a handpiece and in communication with a surgical console, the sensor, the handpiece and the surgical console associated with an ophthalmic surgical system, wherein each of the plurality of pressure values indicates an intraocular pressure (IOP) measurement taken during a surgical procedure;
calculating an overall measurement based on the pressure values; and
displaying on a display device a graphic representation of the overall measurement, wherein the overall measurement illustrates the eye chamber pressure stability.
